(19) United States
(12) Patent Application Publication
(10) Pub. No.: US 2025/0000453 A1
(43) Pub. Date: Jan. 2, 2025

(54) SYSTEMS AND METHODS TO IMPROVE PATIENT SELF-ADMINISTRATION

(71) Applicant: NOBLE INTERNATIONAL, LLC, Orlando, FL (US)

(72) Inventors: Jeffery A. LETTMAN, Orlando, FL (US); Jeff BAKER, Orlando, FL (US); Craig BAKER, Lebanon, OH (US); Isabelle MENARD, Nyack, NY (US); Chris EVANS, Orlando, FL (US); Tim MCLEROY, Orlando, FL (US); Ryan LAW, Altamonte Springs, FL (US); Joseph REYNOLDS, Belle Isle, FL (US); Randy CARDOZA, Winter Garden, FL (US)

(21) Appl. No.: 18/709,666

(22) PCT Filed: Nov. 14, 2022
(86) PCT No.: PCT/US22/49808

§ 371 Date: May 13, 2024

Related U.S. Application Data
(60) Provisional application No. 63/278,810, filed on Nov. 12, 2021.

(51) Int. Cl. A61B 5/00; A61B 5/11; A61B 5/16; G16H 50/30

(52) U.S. Cl. CPC A61B 5/4833; A61B 5/0022; A61B 5/1112; A61B 5/165; A61B 5/4839; G16H 50/30

(57) ABSTRACT

Disclosed is a system for providing personalized self-medication, onboarding, training and improving medication adherence. The system includes a patient computer system comprising at least one processor and at least one memory component: a display, an optional input device, and an optional pointing device, a server connected to the patient computer system by a network link, the server comprising at least one processor and at least one computer readable medium wherein at least one computer readable medium comprises instructions that when executed by at least one processor, cause at least one processor to perform acts comprising: (i) responsive to patient input data, producing a patient personalized assessment; (ii) formulating a customized intervention for the patient based on the patient input data and/or personalized assessment; and (iii) presenting the patient personalized assessment and/or customized intervention on the display.

### BACKGROUND

Patients diagnosed with a chronic disease such as diabetes, rheumatoid arthritis, multiples sclerosis, asthma/COPD are often prescribed medications using drug delivery devices for long-term treatment. Given the trend towards complex, self-administered medications, the patient/caregiver may forget critical steps required to correctly operate the drug delivery device, increasing the likelihood of errors. Consequently, pharmaceutical companies, specialty pharmacies and hub companies have developed patient support programs (alternatively patient assistance programs) to aid patients in self-administration, provide medication and disease-state educational materials, coordinate care and facilitate insurance-related processes. Of the research that is available involving these patient programs, stakeholders (HCPs, patients, caregivers) evidence has consistently demonstrated positive impacts on humanistic and clinical outcomes.

### SUMMARY

In one embodiment, provided is a system for providing personalized self-medication onboarding, training and improving medication adherence. Within the drug delivery industry, the first few months of a patient's treatment is commonly referred to as the onboarding stage. During the onboarding process, healthcare providers (HCPs) introduce patients to their therapies and drug delivery devices and are intended to train patients on proper self-injection prior to sending them home with their devices and other related materials. The system may include a patient computer system comprising at least one processor and at least one memory component; a display, an optional input device, and an optional pointing device, a server connected to the patient computer system by a network link, the server comprising at least one processor and at least one computer readable medium. The at least one computer readable medium comprises instructions that when executed by the at least one processor, cause the at least one processor to perform acts including:
(i) responsive to patient input data, producing a patient personalized assessment;
(ii) formulating a customized intervention for the patient based on the patient input data and/or personalized assessment; and
(iii) presenting the patient personalized assessment and/or customized intervention on the display.

In a specific embodiment, the patient input data includes posology data, environmental factors, treatment history information, training usage data, training effectiveness data, emotional state information, disease state information, physiological data, treatment adherence data, diagnostic data, location data, temporal data, and/or treatment effectiveness data. The patient input data, or portions thereof, may also be transmitted to Health care providers, health care payers, or pharmacies. In a specific example, the patient computer system includes a network link to a local area network that communicates to the server via an internet connection. In a specific version, the patient computer system is a mobile device that wirelessly communicates with the server via an internet connection. In a specific example, producing a patient personalized assessment includes producing a personalized assessment of patient training needs. In another specific example, producing a patient personalized assessment includes producing a personalized assessment of patient treatment needs. In another example, the customized intervention includes a customized training intervention. One benefit of the system described above is that it processes multiple data streams to provide personalized interventions, and importantly, provides such interventions at the most opportune or effective time (i.e. when the patient is most receptive or would most benefit).

In certain examples, a patient personalized assessment includes producing a personalized assessment of patient emotional needs. Also, the customized intervention may include a customized training intervention and/or customized treatment intervention.

In another embodiment, the patient input data comprises training usage data and the at least one memory component of the server further comprises instructions to cause the at least one processor of the server to present on the display a motivational element to the patient in response to training usage data. The patient input data may include treatment effectiveness data, and the at least one processor is instructed to present on the display a motivational element to the patient in response to treatment effectiveness data.

In certain embodiments, the patient computer system communicates with an accessory device and/or a medicament delivery device via a wired or wireless connection. The accessory device may include a training device, or a delivery assistive device or a device with a combination of such functions. The accessory device or medicament delivery device typically includes at least one sensor. In a specific example, the at least one sensor detects operation of the accessory device and/or medicament delivery device, and accordingly, the patient input data may include information about the operation of the accessory device and/or medicament delivery device In a another embodiment, the server includes a storage device onto which training videos or training text is stored and wherein the at least one memory component comprises instructions causing the at least one processor of the server to present the training video(s) or training text on the display. In a specific embodiment, the server records access and/or presentation of the training videos or training text to the display. Optionally, the at least on memory component causes the at least one processor to present a motivational element on the display in response to access of training video(s) or training text.

In a further embodiment the server includes a storage device comprising a plurality assessment variables stored thereon and wherein the at least one memory component of the server comprises instructions that causes the at least one processor to select, in response to patient input data, a combination of assessment variables from the plurality to form the personalized assessment and to present the personalized assessment on the display.

Further, the server may include a storage device comprising a plurality of training variables stored thereon and wherein the at least one memory component of the server comprises instructions that causes the at least one processor to select, in response to patient input data, a combination of intervention variables from the plurality of intervention variables to form the customized intervention and to present the personalized assessment on the display.

In another embodiment, the server includes a storage device comprising a plurality of motivational element variables stored thereon and wherein the at least one memory component of the server comprises instructions that causes the at least one processor to select, in response to patient input data, one or more motivational element variables from the plurality of motivational element variables to form the motivational element and to present the motivational element on the display. In another example, the server comprises a storage device comprising a plurality of surveys stored thereon and wherein the at least one memory component of the server comprises instructions that causes the at least one processor to select, in response to patient input, a survey and to present the survey on the display.

In a further embodiment, the at least one sensor of the accessory device is a physiological sensor that is integral to or connected to an accessory device that comprises circuitry to convey information to the server directly through a wireless connection, or indirectly through the patient computer system or delivery device. The physiological sensor may be connected wirelessly to the accessory device.

In a specific embodiment, the at least one sensor associated with the accessory device communicates with the patient computer system, accessory device and/or delivery device. The at least one sensor may include one or more of a light sensor, fluid flow rate sensor, strain gauge sensor, temperature sensor, pressure sensor, tilt sensor, force sensor, level sensor, contact sensor, photoelectric sensor, magnetic sensor, ultrasonic sensor, electrochemical sensor, acceleration sensor, moisture sensor, humidity sensor, speed sensor, inductive sensor, capacitive sensor, physiological sensor, temporal sensor, geolocation sensor, or orientation sensor. Also, in some embodiments, the at least one sensor includes two or more sensors. In alternative embodiments, the at least one sensor is integral to the patient computer system, and/or is wirelessly connected to the patient computer system; is integral to the accessory device, and/or wirelessly connected to the accessory device; is integral to the delivery device, and/or wirelessly connects to the delivery device. In certain embodiments, the at least one sensor detects errors of mechanical operation of the accessory device and/or delivery device or errors in patient use of the accessory device and/or delivery device.

In certain embodiments, the accessory device comprises a display to convey personalized assessment and/or customized intervention to the patient.

Information of the at least one sensor may be sent to the server via the patient computer system, the accessory device, and/or the delivery device. In an embodiment, the personalized assessment and/or customized intervention is based, at least in part, on information from the sensor. In a specific embodiment, the at least one sensor is a geolocation sensor that detects a patient's location during use of an accessory device and/or delivery device. Alternatively, the at least one sensor comprises a temporal sensor that detects a time at which a patient uses the accessory device and/or delivery device. Information from the geolocation sensor or temporal sensor, or both, may be sent to the server. The sensor may further include a physiological sensor, that is helpful to track disease state and symptoms. Alternatively, an additional sensor separate to the geolocation sensor and temporal sensor is provided that detects operation of the accessory device and/or delivery device.

The accessory device or patient computer system may also include a camera.

According to another embodiment, provided is a method for providing personalized self-medication training and improving medication adherence executed on a server comprising at least one processor and at least one memory component. The method involves receiving patient input data from a patient computer system, the patient computer system comprising at least one processor, at least one memory component and a display; producing a patient personalized assessment by selecting from a plurality of assessment variables stored on at least one storage device; formulating a customized intervention for the patient based on the patient input data or personalized assessment; and presenting the patient personalized assessment and/or customized intervention on the display. The patient input data may include posology data, environmental factors, treatment history, physiological parameters, training usage data, training effectiveness data, emotional state, disease state, treatment adherence, diagnostic data, and/or treatment effectiveness data.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided. It should be borne in mind that all patents, patent applications, patent publications, technical publications, scientific publications, and other references referenced herein are hereby incorporated by reference in this application in order to more fully describe the state of the art to which the present invention pertains.

### System Overview

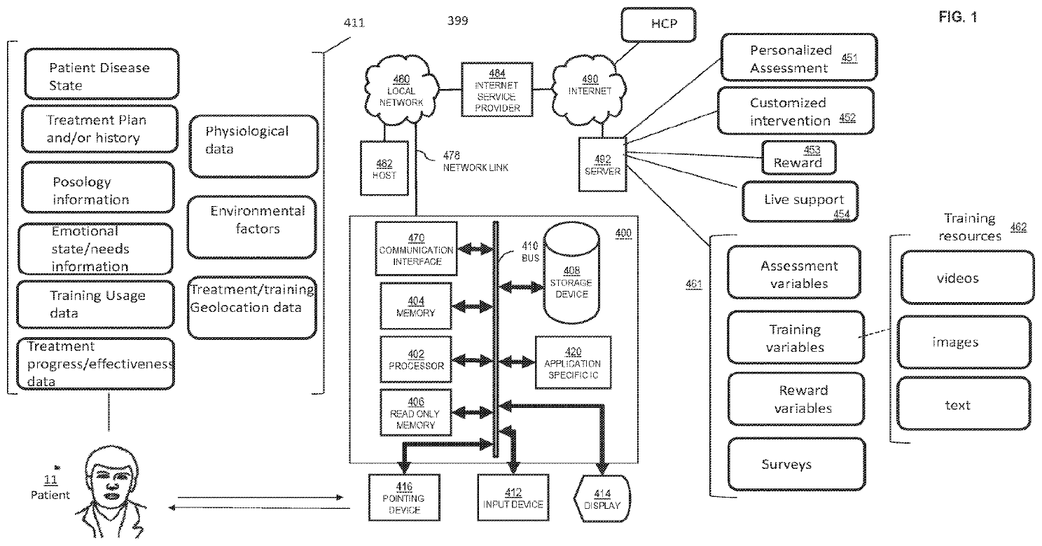

FIG. 1 is a block diagram that illustrates a personalized self-medication training and improving medication adherence system 399 with a computer architecture upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments. such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random-access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read-only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power. Reference to memory component may include at least one of memory 404, ROM 406 or storage device 408. Reference to storage device includes ROM

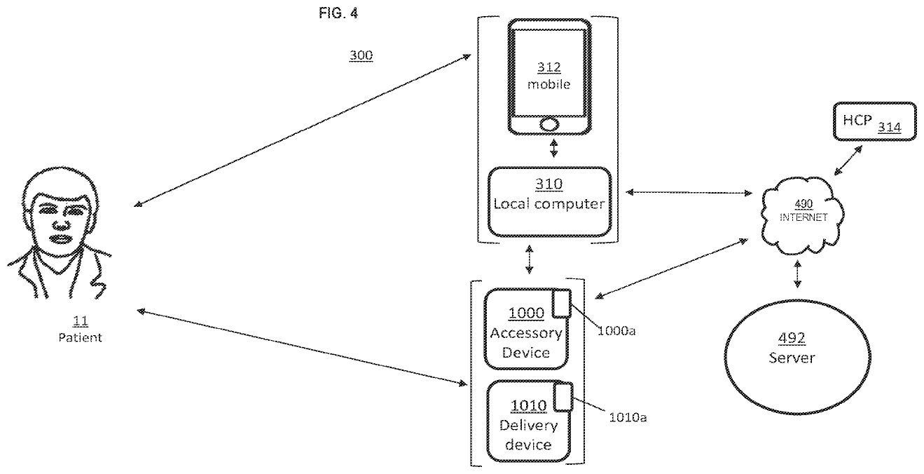

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414. As will be discussed below with respect to FIGS. 4-6, the patient may interface with the computer system 400 via a mobile device 600, accessory device 1000, or medicament delivery device 1010, which may communicate with each other as well. Alternatively, the mobile device 600, accessory device 1000 and/or delivery device 1010 may communicate with a remote server 492 via the internet 490 without having the need for computer system 400.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners, and external disks. In general, the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals, that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. In other embodiments computer-readable medium includes read only memory (ROM) 406. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC *420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing image or video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received, or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

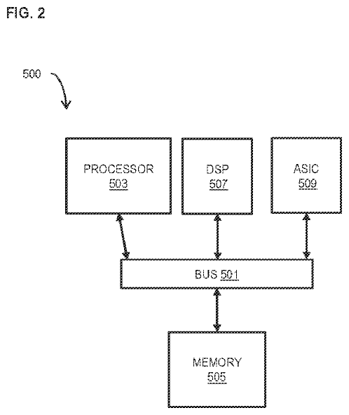

The server 492 may comprise computer elements similar to the patient computer system 400 such as at least one processor and at least one memory component. FIG. 2 illustrates a general chip set 500 that can be implemented in the patient computer system 400 or in the server 492. Chip set 500 is programmed to perform one or more steps of a method described herein and includes, for instance, the processor and memory components described with respect to FIG. 1 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores. Alternatively, or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

The chipset 500 may be implemented in one of many computing devices including, but not limited to, a smartphone, a personal or laptop computer, a tablet computer, an internet portal, or an internet application.

Referring back to FIG. 1, the server 492 has stored thereon, typically a storage device similar to 408, various information modules 461 containing categories of variables from which a processor 503 can select and combine to formulate a personalized assessment 451, customized intervention 452 or motivational element 453. Examples of motivational elements presented to the patient 11 include game points, digital badges or medals, monetary incentives or tangibles of assorted value (i.e. beverages, clothing, merchandise, etc.). The modules 461 shown include assessment variables, training variables, motivational element variables and surveys. Surveys may take the form of a treatment questionnaire to help patients identify the best medication based on device type, product features, prior Rx history, injection frequency, etc. Surveys may also take the form of a follow-up survey to monitor comprehension and recall over time including topics like injection frequency, injection hold time, etc.

Also shown are training resources 462 that can be included as part of a customized intervention 452 or presented to the display 414 in response to a patient 11 request. The server 492 is also programmed so as to facilitate live support 454 (e.g., live text or video) to the patient 11 via computer 400 and display 414.

The patient data 411 is inputted manually or automatically into the patient computer system. The patient data 411 includes information useful for formulating a personalized assessment 451 and customized intervention 452, and includes, but is not limited to, posology data, environmental factors, treatment plan/history, physiological data, training usage data. training effectiveness data, emotional state/needs information, disease state, treatment adherence, diagnostic data, treatment/training location and/or treatment progress/effectiveness data. Emotional state can be inputted by the patient. Numerical rating scale, for example a respondent may be asked to rate a confidence on a scale from 1 (“not confident at all”) to 5 (“extremely confident”). Allows for the capture of emotional state with specificity. The uniform scale makes it easy to compare across different respondents. Digital journaling or Positive and Negative Affect Schedule (PANAS) can be inputted as patient input data and can help to correlate emotions with symptoms and triggers. It also helps to establish a routine.

Environmental factors can be inputted as patient input data. Environmental factors may be in the form of indirect measures of a user's emotional state by reviewing activities and understanding their environment. Geolocational data is useful in indirectly assessing a user's emotional state, which includes information about patient current location (home, store, leisure, work, restaurant, health, gym, religious, traveling, etc.), regularity of patient pattern of movements, known as circadian movement, percentage of time spent in transit, or percentage of time spent outdoors or conducting physical activity. Geolocation data can be obtained generally or timed for recording during treatment or training.

Web activity tracking may help to establish a user's emotional state. There is a plethora of information related to a user's online behavior that may potentially reveal insights into their emotional state: amount of time spent online or using social media. Further, parameters that involve interfacing with a system embodiment can be tracked and recorded to determine emotional state. These include typing speed, typing mistakes, usage of special characters, and typing duration or pointing device 416 usage.

Treatment progress/effectiveness data may take several forms that can be manually inputted or tracked by a system embodiment. The above information regarding emotional state/needs can be used to devise algorithms to generate a customized emotional needs intervention. Factors to consider in these interventions is provided in Table 1 below. Having access to the emotional state of patients, in combination with training effectiveness data, can serve to devise algorithms which provide the patients with the right information and resources at the most impactful time.

Treatment progress/effectiveness data can be obtained as follows:
Direct measurement:
Direct measurement of auto-injector usage.
accessory device usage.
track correct trainer usage.
Smart pill bottles, inhalers, manual injection devices, etc.

### (i) Medication Possession Ratio

Sum of days supply for a given period dispensed to patient divided by the number of days in the period.

### (ii) Portion of Days Covered

Number of days covered in each period divided by the number of days in the period
More accurate than Medication Possession Ratio because it removes the potential for dispensing overlap.
URAC (gold standard for specialty pharmacy accreditation) uses Portion of Days Covered for their adherence measures.
   Indirect reporting
(i) Self-reporting of injection.
(ii) Care coordinator check-ins via text/call/email/etc.
(iii) Logging of EMR/EHR health data and pharmacy information (manual or automated).
(iv) Monitor patient's copay card data.
Co-pay card programs are patient-based programs designed by manufacturers to assist commercially insured and cash paying patients in affording their medications.
(v) Monitor script fill-rates
Medication name, prescription dosage, dosing schedule, etc.
Number of additional fills over baseline via backend integration from copay provider.
Gap days between fills
90-day filled prescription data
(vi) Number of days on therapy
Appointment Abandonment/Attendance
(vii) Attitudes toward Treatment Adherence
Patient wellbeing/discomfort, belief about damage from medication, disease treatment complications, barriers/facilitators to medication, accessibility of treatment and health services, doctor-patient agreement about treatment, etc.
(viii) Pictures and scanning of used autoinjectors and new drug cartons as adherence and coaching proxies.
   Treatment Effectiveness Can be monitored as follows:
   Self-reported tracking of symptoms in a digital diary.
(i) Telemedicine based on self-measurement of disease activity could be a key source of treatment data. Previous publications in various medical fields have shown that it is possible to safely telemonitor patients while reducing the number of outpatient clinic visits.
   Documentation from HCP following virtual or in-office visits.
(i) Hospitalizations, laboratory tests (e.g., blood tests and glycemic control). BMI, heart rate, blood pressure, daily log of symptoms and medication administration, peak flow rate (for respiratory medications), quality-of-life surveys, lifestyle change surveys, and satisfaction surveys.
   Specialized remote assessments of treatment, examples include:
(i) AI enabled teledermatology for plaque psoriasis (Koller et al, 2011)
(ii) Remote Digital Neurological Assessment for Patients with MS (Pham et al, 2020)
   Generic Remote Diagnostic tools:

As will be discussed with respect to FIGS. 4-6, the patient input data may be communicated to the patient computer system directly or indirectly via an accessory device, physiological parameter component or a medicament delivery device. Indirect communication may occur through an intervening computing device such as a mobile terminal. The server 492 or system 400 may also include a medical translation feature for medication guides and IFUs (ex. Language translation apps), for example animated IFU videos for various steps. Picture/scanning of drug carton to streamline the enrollment process and connect patients with the right information.

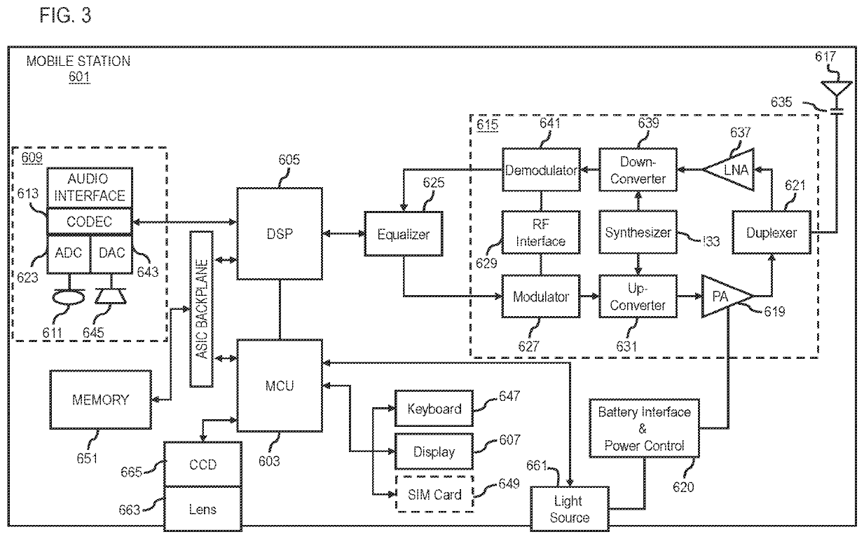

FIG. 3 is a diagram of exemplary components of a mobile terminal 600 (e.g., cell phone handset) for communications, which is capable of operating in the system of FIG. 2C, according to one embodiment. In some embodiments, mobile terminal 601, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 603, a Digital Signal Processor (DSP) 605, and a receiver/transmitter unit including a microphone gain control unit and a speaker gain control unit. A main display unit 607 provides a display to the user in support of various applications and mobile terminal functions that perform or support the steps as described herein. The display 607 includes display circuitry configured to display at least a portion of a user interface of the mobile terminal (e.g., mobile telephone). Additionally, the display 607 and display circuitry are configured to facilitate user control of at least some functions of the mobile terminal. An audio function circuitry 609 includes a microphone 611 and microphone amplifier that amplifies the speech signal output from the microphone 611. The amplified speech signal output from the microphone 611 is fed to a coder/decoder (CODEC) 613.

A radio section 615 amplifies power and converts frequency in order to communicate with a base station, which is included in a mobile communication system, via antenna 617. The power amplifier (PA) 619 and the transmitter/modulation circuitry are operationally responsive to the MCU 603, with an output from the PA 619 coupled to the duplexer 621 or circulator or antenna switch, as known in the art. The PA 619 also couples to a battery interface and power control unit 620.

In use, a user of mobile terminal 601 speaks into the microphone 611 and his or her voice along with any detected background noise is converted into an analog voltage. The analog voltage is then converted into a digital signal through the Analog to Digital Converter (ADC) 623. The control unit 603 routes the digital signal into the DSP 605 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 625 for compensation of any frequency-dependent impairments that occur during transmission though the air such as phase and amplitude distortion. After equalizing the bit stream, the modulator 627 combines the signal with a RF signal generated in the RF interface 629. The modulator 627 generates a sine wave by way of frequency or phase modulation. In order to prepare the signal for transmission, an up-converter 631 combines the sine wave output from the modulator 627 with another sine wave generated by a synthesizer 633 to achieve the desired frequency of transmission. The signal is then sent through a PA 619 to increase the signal to an appropriate power level. In practical systems, the PA 619 acts as a variable gain amplifier whose gain is controlled by the DSP 605 from information received from a network base station. The signal is then filtered within the duplexer 621 and optionally sent to an antenna coupler 635 to match impedances to provide maximum power transfer. Finally, the signal is transmitted via antenna 617 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 601 are received via antenna 617 and immediately amplified by a low noise amplifier (LNA) 637. A down-converter 639 lowers the carrier frequency while the demodulator 641 strips away the RF leaving only a digital bit stream. The signal then goes through the equalizer 625 and is processed by the DSP 605. A Digital to Analog Converter (DAC) 643 converts the signal and the resulting output is transmitted to the user through the speaker 645, all under control of a Main Control Unit (MCU) 603 which can be implemented as a Central Processing Unit (CPU) (not shown).

The MCU 603 receives various signals including input signals from the keyboard 647. The keyboard 647 and/or the MCU 603 in combination with other user input components (e.g., the microphone 611) comprise a user interface circuitry for managing user input. The MCU 603 runs a user interface software to facilitate user control of at least some functions of the mobile terminal 601 as described herein. The MCU 603 also delivers a display command and a switch command to the display 607 and to the speech output switching controller, respectively. Further, the MCU 603 exchanges information with the DSP 605 and can access an optionally incorporated SIM card 649 and a memory 651. In addition, the MCU 603 executes various control functions required of the terminal. The DSP 605 may, depending upon the implementation, perform any of a variety of conventional digital processing functions on the voice signals. Additionally, DSP 605 determines the background noise level of the local environment from the signals detected by microphone 611 and sets the gain of microphone 611 to a level selected to compensate for the natural tendency of the user of the mobile terminal 601.

The CODEC 613 includes the ADC 623 and DAC 643. The memory 651 stores various data including call incoming tone data and is capable of storing other data including music data received via, e.g., the global Internet. The software module could reside in RAM memory, flash memory, registers, or any other form of writable storage medium known in the art. The memory device 651 may be, but not limited to, a single memory. CD, DVD, ROM, RAM, EEPROM, optical storage, magnetic disk storage, flash memory storage, or any other non-volatile storage medium capable of storing digital data.

An optionally incorporated SIM card 649 carries, for instance, important information, such as the cellular phone number, the carrier supplying service, subscription details, and security information. The SIM card 649 serves primarily to identify the mobile terminal 601 on a radio network. The card 649 also contains a memory for storing a personal telephone number registry, text messages, and user specific mobile terminal settings.

In some embodiments, the mobile terminal 601 includes a digital camera comprising an array of optical detectors, such as charge coupled device (CCD) array 665. The output of the array is image data that is transferred to the MCU for further processing or storage in the memory 651 or both. In the illustrated embodiment, the light impinges on the optical array through a lens 663, such as a pin-hole lens or a material lens made of an optical grade glass or plastic material. In the illustrated embodiment, the mobile terminal 601 includes a light source 661, such as a LED to illuminate a subject for capture by the optical array, e.g., CCD 665. The light source is powered by the battery interface and power control module 620 and controlled by the MCU 603 based on instructions stored or loaded into the MCU 603.

FIG. 4 shows a general setup of a system embodiment 300 for providing personalized self-medication training and improving medication adherence. The system embodiment 300 comprises an accessory device 1000 and/or delivery device 1010 that communicates information actively or passively, or a combination of both, to a local computer 310 and/or mobile device 312. Active communication involves a wired or wireless connection wherein data is transmitted to the local computer 310 or mobile device 312. Passive communication would take the form of information such as sounds, lights, images or motion that is detectable, for example, by a sensor or camera associated with the local computer 310 or mobile device 312. The local computer 310 or mobile device sends patient input data to a server 492 via an internet connection 490. Also, the system 300 may include a communication with a health care provider (HCP) 314. Determination of proper usage during in-office visits, as assessed and logged by HCP, as well as HCP in-office visits. The HCP can also access the system to provide health coaching services. In another embodiment, the system 300 may include a communication with a medicine supplier (e.g., a pharmaceutical company) which will provide valuable information about the patient needs and habits. This information can be utilized to modulate medicaments and/or delivery devices to improve and enhance the patient experience.

In an alternative embodiment, the accessory device 1000 and/or delivery device 1010 comprise circuitry that allow them to communicate with server 492 via internet 490 without communicating with either a local computer 310 or mobile device 312. The accessory device 1000 and/or delivery device 1010 may also include components such as a display and/or user interface 1000a, 1010a, respectively to provide information to the user. In one example, the patient personalized assessment or customized intervention, or both, may be communicated to the accessory device 1000 or delivery device 1010, or both. For example, the personalized assessment or customized intervention, or both, may be presented on a display 1000a or 1010a present on either the accessory device 1000 or delivery device 1010. The accessory device 1000 or delivery device 1010 may include circuitry and one or more sensors to detect errors either in mechanical operation of the delivery device or accessory device, or errors in operation of either device by the patient, collectively referred to herein as operational information.

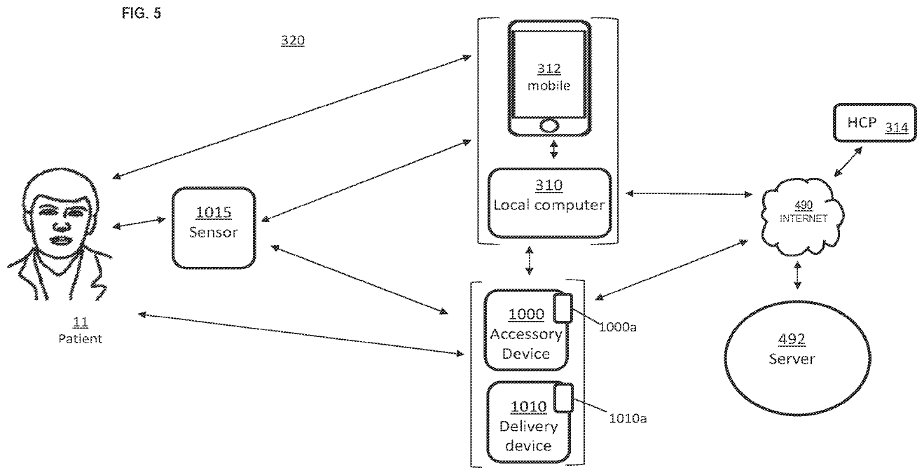

FIG. 5 shows another system embodiment 320 for providing personalized self-medication training and improving medication adherence. The system embodiment 320 includes an accessory device 1000 and/or delivery device 1010 that communicates information actively or passively, or a combination of both, to a local computer 310 and/or mobile device 312, as described in relation to FIG. 4 above. FIG. 5 shows the implementation of a sensor 1015. The sensor 1015 may be independent of or integral to the accessory device 1000 or delivery device 1010. A sensor being integral with respect to a device means that it is rigidly attached to the device. Examples of sensors include, but are not limited to, a camera, sound detecting component, a physiological sensor, temporal sensor and/or a geolocation sensor. Alternatively, the sensor 1015 is integral to the local computer 310 or mobile device 312. As described above, the accessory device 1000 and/or delivery device 1010 comprise circuitry that allow them to communicate with server 492 via internet 490 without communicating with either a local computer 310 or mobile device 312. As noted above with respect to FIG. 4, the accessory device 1000 and/or delivery device 1010 comprise circuitry that allow them to communicate with server 492 via internet 490 without communicating with either a local computer 310 or mobile device 312. The accessory device 1000 and/or delivery device 1010 may also include components such as a display and/or user interface 1000a or 1010a, respectively, to provide information to the user. In one example, the patient personalized assessment or customized intervention, or both, may be communicated to the accessory device 1000 or delivery device 1010, or both. For example, the personalized assessment or customized intervention, or both, may be presented on a display 1000a or 1010a present on either the accessory device 1000 or delivery device 1010. The implementation of an accessory device 1000 or delivery device 1010 that is able to sense and acquire information from the user coupled with connectivity to the server 492, and then able to present information back to the user, provides a unique and beneficial closed loop system between the patient 11 and the system 320.

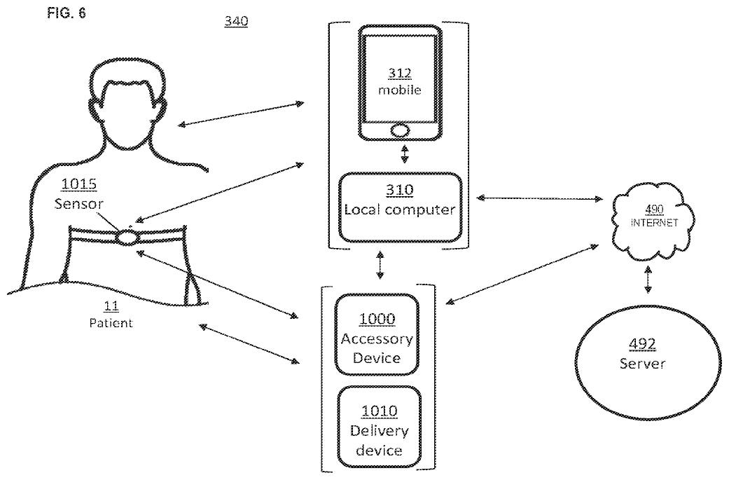

Turning to FIG. 6, shown is a system 340 for providing personalized self-medication training and improving medication adherence. The system embodiment 340 includes an accessory device 1000 and/or delivery device 1010 that communicates information actively or passively, or a combination of both, to a local computer 310 and/or mobile device 312, as described in relation to FIG. 4 above. FIG. 6 shows the implementation of a sensor 1015 where the sensor is a wearable physiological sensor that senses a physiological parameter. The term “physiological parameter” refers to any measurable or detectable physiological indicator of a subject. Examples of physiological parameters include, but are not limited to heart rate, heart rate variability (HRV), temperature, neural activity (e.g. EEG, ECG), respiratory sinus arrhythmia (RSA), galvanic skin response (GSR) respiratory hippus variability (RHV) and gastric movement, e.g., via use of electrogastrography (EGG) or other techniques to determine gut motility. Physiological parameters may also include respiration, respiratory variability, blood pressure, blood pressure variability, pulse wave size, slope, etc, pulse variability, body tonicity (alteration in body movement), tremor/repetitive motor activity, pilo-erection, reproductive organ behavior, EGG: Electrogastrogram (and other measures of gastric movement), fMRI, EEG and other measures of direct neural behavior. Physiological parameters can also include skin temperature change or skin conductance. The sensor 1015 may be independent of or integral to the accessory device 1000 or delivery device 1010. Alternatively, the sensor 1015 is integral to the local computer 310 or mobile device 312. As described above, the accessory device 1000 and/or delivery device 1010 comprise circuitry that allow them to communicate with server 492 via internet 490 without communicating with either a local computer 310 or mobile device 312. In addition, the sensor 1015 can sense other behaviors such as voice characteristics, facial expressions or posture. Such observations can be tracked and recorded in conjunction with treatment or training sessions, or while they are using a system embodiment described herein (e.g. 300. 320, 340 or 399).

### Accessory Devices and Delivery Devices

Exemplary embodiments of an accessory device 1000 can be implemented to educate users on the proper operation and usage of a delivery device. The accessory device 1000 may be a device used to simulate medicament delivery referred to herein as a training device. The accessory device may be an assistive device that is used in conjunction with a delivery device 1010 to assist in delivery of a medicament by instructions to the user or real-time feedback such as via a signal output component; such device referred to herein as an assistive device. The accessory device can be used to make prospective and current users of delivery devices feel more comfortable and confident in self-administration (or administration to others) of medicaments and can help users understand the proper steps of medicament delivery. Exemplary embodiments of a training device can be used by a user before the user administers a medication by way of, for example, an auto-injector by way of using an actual automatic injection device corresponding to the automatic injection training device and/or can be used as needed or desired by the user. Other exemplary embodiments of training devices pertain to manual injection devices and manual injection training devices used by the user, respiratory inhaler trainers and respiratory inhaler drug delivery devices, in non-limiting examples.

In one example, the accessory device may be equipped to produce visual stimuli or feedback (visual output) can be generated mechanically or electronically. An example of a mechanically generated visual stimulus is a plunger moving past an inspection window in an autoinjector or prefilled syringe delivery device or a shroud extending from an injection device. An example of an electronically generated visual stimulus is one or more LED's blinking, an LCD display showing an icon, or key steps in the process of administration being highlighted on a screen in the order required for proper administration of medicament, in non-limiting examples. A visual output as disclosed herein includes but is not limited to a light, a display, a colorometric display system, a change in position of the device or any other type of visual cue to the user of the container and/or device. The visual output is associated with the delivery device or with the medicament training container; therefore it may be disposed on either portion of the system or provided in connection with the system either by a wire or wirelessly.

Additional visual outputs that may be incorporated into the system herein may include display devices having one or more layers of material having a light transmission region, a unit of information to be highlighted, and a light blocking region; and a backlight unit having a flexible, planar waveguide body, a light source configured to direct light into the waveguide body, and at least one light director associated with a portion of the waveguide body so as to direct light transversely to a plane of the waveguide body. The directed light travels through the light transmission region, and the directed light is directed toward the unit of information to be highlighted as provided in International Application No. PCT/US11/26976 and US National Stage Application Serial No. 13/582,560 which claim the benefit of U.S. Provisional Application Ser. No. 61/310,081, which are incorporated by reference in their entireties herein. The unit or units of information to be highlighted may include the stepwise instructions for administering the medicament to a user and may also provide the duration of each step by way of highlighting each step for a predetermined amount of time such that the user can follow the precise timing of each step in the sequence.

Auditory stimuli or feedback (audio output) can also be generated mechanically or electronically. An example of a mechanically generated auditory stimulus is the “click” that can be heard if two parts of a device interlock. An example of an electronically generated auditory stimulus is a beeper or a speaker that plays spoken instructions. An audio output as disclosed herein can be generated mechanically or electronically, for example, and includes but is not limited to music, a sound, a beep, a series of beeps music or sounds, a mechanical sound including clicking, the movement of one or more parts of a delivery device relative to one another, or a sound replication of operation or behavior of a drug delivery device containing medicament. These auditory stimuli, such as two parts of a device interlocking can be picked up by a microphone of the system. The microphone may be associated with the accessory device in a non-limiting example, and may receive audio input from the delivery device as described above during use of the system. The system can then identify whether or not the device was used correctly (i.e., whether a step was performed correctly or in the correct order, for example). A combination of both visual and auditory output may include a video tutorial providing instructions to a user on proper administration of the medicament or use of the training device, for example.

Somatic stimuli or feedback, also called somatosensory stimuli or tactile feedback, is typically generated mechanically. There are a large number of somatic stimuli, particularly with reference to the delivery device, such as actuation forces, abrasion resistance, frictional forces. spring compression, the feel of a click if two parts interlocking, surface texture, vibrations, weight sensation, and any other similar stimuli or feedback known to those of skill in the art.

A “predetermined value” as used herein, for example, includes but is not limited to a value or range of values relating to an event involving use or operation of the device. These may include, but are not limited to thresholds, ceilings, baselines or range values that are desired or undesired for a particular event. Examples of predetermined values include, but are not limited to, a predetermined orientation value, predetermined time value, or a predetermined contact value, in addition to other predetermined values described herein refers to a value that is used as a reference value in relation to a value, signal, or indication that is detected by, for example, a sensor of the delivery device. Predetermined value may include an optimal value, or a sub-optimal value, or any value there between, or any combination thereof. The term “value” as used herein, may refer to a specific value or a range of values.

In one example, a predetermined perpendicularity value may include a 90 degree angle between the device and a target region for the delivery device, an additional predetermined perpendicularity value may include a 10 degree angle between the device and a target region for the device. At either predetermined perpendicularity value, or at any value there between, a signal output component may be initiated. The signal output component may therefore be an error message or a congratulatory message, for example. This signal output component may be initiated from the delivery device and/or the medicament training container.

The term “condition” as used herein includes but is not limited to one or a combination of a user input, a status of the delivery device or the medicament training container, anything that is sensed by the device or container, correct or incorrect stepwise activities, usage of the device over time, among other conditions.

The term “error condition” as used herein includes but is not limited to one or a combination of a condition pertaining to a mistake by the user in using the device, whether the mistake is incorrect positioning or contact between the device and the user, or whether the mistake is an out of order step, a step that exceeds or fails to meet predetermined time value (such as an undue pause during or between steps, or insufficient time for conducting a step or transition between steps). Error conditions may also include errors of the device itself or of the container, including low or lack of power or failure to operate as intended.

The term “fluid” as used herein may include any type of fluid, including but not limited to liquid or gas. The fluid may specifically include a liquid, powder or aerosol medicament, air flowing to or from the user, or liquid coming from the user, in some non-limiting embodiments.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. For example, “a sensor” may denote the presence of at least one sensor; however, multiple sensors may be contemplated unless otherwise specifically stated. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one clement from another.

Reference has been made above to a sensor 1015. The sensor may be independent to the accessory device 1000 or delivery device 1010 or may be integral to one or the other. Sensors that may be integral to the accessory device 1000 or delivery device 1010 may include but are not limited to, light sensors, fluid flow rate sensors, strain gauge sensors, temperature sensors, pressure sensors, tilt sensors, force sensors, level sensors, contact sensors, photoelectric sensors, magnetic sensors, ultrasonic sensors, electrochemical sensors, acceleration sensors, moisture sensors, humidity sensors, speed sensors, inductive sensors, capacitive sensors, physiological sensor, temporal sensor, geolocation sensor, and orientation sensors. Some of these sensors may require a supply of voltage. The medicament system may include one or more of the sensors described herein, for example, a contact sensor may be used to detect removal or placement of the smart device onto the delivery device, for example, or removal or placement of the delivery device onto the user, in another example. In a further example, two or more contact sensors, more preferably thee contact sensors may be used to detect perpendicularity of the delivery device relative to a target area of a patient. An example of an inductive sensor includes material embedded in or associated with the delivery device, wherein said embedded or associated material proportionally changes the magnetic field of the inductive sensor which may be associated with or embedded in the smart device, in one non-limiting example, depending on its distance away from the inductive sensor. The sensor then outputs a variable electrical signal based on the distance between the embedded or associated material and the inductive sensor.

The tactile or vibration component may include a vibration system, which provides a vibratory sensation. In one embodiment, this vibrator system may include a motor, in a non-limiting example a small DC motor, a gear and a weight. The weight may be mounted off-center on the gear, such that when the motor spins, the gear/weight combination at 100 to 150 rpm for example, the off-center mounting creates a vibration. In another embodiment, a linear transducer may be used. In a further example, an eccentric motor can be used to provide an oscillation or vibration.

Embodiments of the accessory device 1000 or delivery device 1010 herein may be provided wherein various components of the system may be incorporated into either one of an accessory device, or delivery device or both. Some components of the system may be in the delivery device and other components of the system may be in the accessory device, in a non-limiting embodiment.

In one embodiment, the accessory device 1000 and/or delivery device 1010 are configured to communicate information about their respective use (i.e., operation). The delivery device 1010 may include a housing, and the accessory device 1000 may include an information detecting and/or receiving component configured to receive information from the delivery device and/or a sending component configured to send information to the delivery device, and optionally at least one of: a) a signal output component; b) a microprocessor; c) a storage medium component; and d) a power source, and wherein the delivery device may be configured to generate information detectable by the accessory device, or transmit information to the accessory device, wherein the accessory device may be configured to detect and/or receive information about the delivery device from the delivery device and provide information about the delivery device and/or a feedback about a use of the delivery device to a system embodiment (e.g. 399, 300, 320 or 340). The accessory device and/or delivery device may further include a transmitter configured to communicate information and/or signals from the delivery device to the accessory device and/or a remote device, and/or receive information and/or signals from a accessory device and/or a patient computer system.

In a further embodiment, the accessory device of the system may be able to receive or obtain information from a remote source as well as send information to a remote source such as the Internet, a database, a telephone, a computer, or other source, in non-limiting embodiments. The information and/or signal sent or received may include information about the delivery device, a user, a medicament, a use of the delivery device, instructions for using the delivery device or the medicament, a physician or other healthcare provider, a hospital, a pharmacy, in non-limiting examples. In a non-limiting embodiment, the accessory device may include a Smartphone which will be described in more detail below.

The signal output component may be configured to output an audio, a visual, a tactile, a gustatory, or an olfactory signal, or a combination thereof, to the user, in non-limiting embodiments.

In another embodiment, the accessory device includes the power source. In another embodiment, the information detecting component may detect movement of the delivery device or components thereof. In some embodiments, the delivery device may include one or more mechanical parts or components that may move relative to one another. The detecting component may detect movement of the delivery device and/or movement of the mechanical components of the delivery device by visual detection in non-limiting embodiments. In addition, or in an alternative embodiment, the detecting component may, detect audio caused by movement of the one or more mechanical parts or components, or movement of components of the delivery device relative to one another by audio detection. The information detected may be provided to a microprocessor and a signal may be provided to a user. Furthermore, the information detected may be stored on the storage medium component. In one non-limiting embodiment, the detecting component may include a camera and/or a motion sensor. In another non-limiting embodiment, the detecting component may include a microphone. In a further, non-limiting embodiment, the detecting component may include both an audio and a visual detection component.

In another embodiment, the receiving component may be configured to receive a signal from the delivery device. The signal may provide information about the delivery device, a medicament associated with the device and/or a use of the delivery device. In a non-limiting embodiment, the receiving component includes a microphone, and the signal received from the delivery device is an audible signal, wherein the microphone is configured to receive the audible signal from the delivery device and/or a mechanical sound produced by a mechanism including one or more mechanical components associated with the housing of the delivery device during use of the delivery device.

In a further embodiment, the delivery device may communicationally connect to the accessory device via a wired or a wireless connection, wherein said connection provides a communication of power and/or information between the delivery device and the accessory device. In some non-limiting embodiments, communication between the delivery device and the accessory device includes one way communication of power and/or information from the accessory device to the delivery device, or from the delivery device to the accessory device, and two-way communication of power and/or information to and from the medicament and accessory devices.

In one non-limiting embodiment, the wireless connection includes a Bluetooth® and/or an RFID (Radio Frequency Identification) technology. In a further embodiment, the RFID technology includes an RFID transponder and an RFID reader. In another non-limiting embodiment, the RFID transponder is associated with the accessory device and the RFID reader is associated with the delivery device, the delivery device includes the power source, such that the RFID reader of the delivery device can power the accessory device by way of the RFID transponder.

The RFID transponders, or tags, described herein may be active, semi-active or passive and may include a microchip and an antenna. The active and semi-active RFID transponders may additionally include a battery, in a non-limiting example.

In another non-limiting embodiment, the RFID transponder is associated with the delivery device and the RFID reader is associated with the accessory device wherein the accessory device includes the power source, and wherein the RFID reader of the accessory device powers the delivery device by way of the RFID transponder.

In a further embodiment, the medicament system is provided wherein one of the delivery device or the accessory device comprises a unique identification component. The unique identification component may include, in non-limiting embodiments, information about a medicament, the delivery device, use of the delivery device, or information about the accessory device, or other information know to one of ordinary skill in the art. The other of the delivery device or the accessory device may include a unique identification reader, wherein the unique identification reader is configured to read information on or associated with the unique identification component, in an embodiment. In one non-limiting embodiment, the unique identification component may include a bar code and the unique identification reader may include a bar code reader.

In another embodiment, the accessory device includes or is configured to receive pre-programmed information. The pre-programmed information may include information about a delivery device, in a non-limiting embodiment. The pre-programmed information may include information about a medicament, a user, a healthcare provider, a healthcare facility, or other information, in other non-limiting embodiments.

In a further embodiment, the accessory device includes circuitry to download or receive information from a database, the Internet, and/or another device. The information may include information about a medicament, a delivery device including instructions for use, contraindication of medicaments, information about a user, medicament dosage information, storage information, prescribing physician information, pharmacy information, manufacturer information, warning information, recall information, in non-limiting embodiments, environmental data, geographical information, time zone information, MET (meteorological) data, storage information, supply chain information, transit data, and/or temperature data.

In a non-limiting embodiment, the accessory device may include the signal output component, the microprocessor, the storage medium component and the power source. In another embodiment, the signal output component may include a speaker, a display, a light, a vibration component, a smell-emitting component, or a temperature-changing component, or a combination thereof.

In a further embodiment, the accessory device may be configured to detect a condition of the delivery device in response to information received by the information detecting and/or receiving component and/or information received via the wired or wireless connection with the delivery device. In a further embodiment, the condition may include an error condition, a correct usage of the system, and/or an input sensed by the system. In another embodiment, the medicament system may provide feedback to a user of the system via the signal output component. This feedback may be based on a condition detected, in a non-limiting embodiment.

In another embodiment, the server 492 or patient computer system (e.g. mobile device 312 or local computer 310) may provide instructions for using the delivery device in a sequence of steps, wherein the microprocessor is configured to control a provision of the instructions for using the delivery device to the user in the sequence of steps and/or to provide an instruction to the user based on information detected and/or received by the information detecting and/or receiving component of the accessory device.

In a further embodiment, the delivery device may include a sensor associated with the delivery device, wherein the sensor may be configured to detect a condition of the delivery device. Information about the condition detected by the sensor may be communicated to the accessory device and/or to a user from the delivery device, in an embodiment. The information that is communicated to the accessory device may further be provided from the accessory device to the user in a non-limiting embodiment.

In another embodiment, the accessory device includes the microprocessor, the storage medium component, the power source, and the signal output component. In a further embodiment of the medicament system, the delivery device may connect to the accessory device via a wired and/or a wireless connection, wherein said connection provides a communication of power and/or information between the delivery device and the accessory device. A communication of power or information can be either one way or two-way. One way communication may occur from the accessory device to the delivery device or vice versa, and two-way communication between the accessory device and the delivery device may occur, in non-limiting embodiments. Communication of power may be from the accessory device to the delivery device in one non-limiting embodiment, from the delivery device to accessory device in another non-limiting embodiment. As aforementioned, the communication may be one way communication or two-way communication between the components of the system. In one particular non-limiting embodiment, information communication is communicated from the delivery device to the accessory device, and power may be communicated from the accessory device to the delivery device.

As aforementioned, the signal output component of the medicament system may include a speaker, a display, a light, a vibration component, a smell-emitting component, or a temperature-changing component, or a combination thereof in non-limiting embodiments. In another embodiment of the medicament system, the delivery device may include a mechanism including one or more mechanical components associated with the housing that move relative to one another to produce a visual, a tactile and/or an audible output detectable by the detecting component. In a further embodiment of the system, the accessory device may be configured to detect a condition of the delivery device in response to information received by the information detecting and/or receiving component and/or information received via the wired and/or wireless connection with the delivery device.

In a further embodiment the medicament system is provided wherein the condition includes an error condition, a correct usage of the system, and/or an input sensed by the system. In still a further embodiment, the system may provide feedback to a user of the system via the signal output component based on the condition detected. In another embodiment, the system may provide instructions for using the delivery device in a sequence of steps, and the microprocessor may be configured to control a provision of the instructions for using the delivery device to the user in the sequence of steps and/or to provide an instruction to the user based on information produced from the sensor of the delivery device.

In yet another embodiment, a medicament system configured to communicate information about a delivery device or about a use of a delivery device, or a combination thereof, to a user, is provided. The medicament system includes a delivery device having a housing, a accessory device comprising a sensor, and an attachment component configured to secure the accessory device to the delivery device. The system may further include in an embodiment, a sending component configured to send information to the delivery device and/or the accessory device, an information detecting and/or receiving component configured to receive information from the delivery device about the delivery device and/or about a use of the delivery device. In embodiments of the system in which the accessory device is attached or secured to the delivery device, the accessory device may not require an information detecting and/or receiving component configured to receive information from the delivery device as the accessory device will sense the condition of the delivery device by virtue of the attachment in non-limiting embodiments. The system may further include a signal output component, a microprocessor, a storage medium component, and a power source, wherein the signal output component is configured to provide an output comprising information about a usage of the delivery device.

In a further embodiment the medicament system is provided wherein the delivery device includes the microprocessor, the storage medium component, the signal output component, and the power source. In still a further embodiment, the delivery device may connect to the accessory device via a wired and/or a wireless connection, wherein said connection provides a communication or transmission of power and/or information between the delivery device and the accessory device. In one non-limiting embodiment, the wireless connection may be short-range wireless connection, such as Bluetooth® connection in one non-limiting example. In a non-limiting embodiment, where a Bluetooth® connection is used a power source may be provided in both the delivery device and the accessory device. The power source may include a battery, in one non-limiting example.

In a non-limiting embodiment of the medicament system, the accessory device may include the microprocessor and the storage module, the accessory device including pre-programmed information about one or more delivery devices and/or instructions for use for one or more delivery device and/or medicaments associated therewith, for example. In a further embodiment, the accessory device may be configured to download or receive information from a database, the Internet, and/or another device, the information may include information about one or more medicaments, one or more delivery devices including instructions for use, contraindication of medicaments, information about one or more users, medicament dosage information, storage information, prescribing physician information, pharmacy information, manufacturer information, warning information, recall information in non-limiting embodiments, environmental data, geographical information, time zone information, MET (meteorological) data, storage information, supply chain information, transit data, and/or temperature data. Such temperature data may include, in embodiments in which the delivery device includes medicament, what temperature the medicament has been kept at and/or a report of temperatures that the delivery device and/or medicament has been subjected to from the manufacturer to the user, for example. Other temperature data may include temperature data indicating required or suggested storage temperatures for medicaments and/or delivery devices.

In another embodiment of the medicament system, the delivery device may include a mechanism including one or more mechanical components associated with the housing that move relative to one another to produce a visual, a tactile and/or an audible output detectable by the detecting component. In one non-limiting example, wherein the delivery device includes a respiratory device or trainer and specifically a metered dose inhaler, for example, the one or more mechanical components of the delivery device may include the components of the metered dose inhaler housing that move relative to one another when the inhaler is actuated.

In a further embodiment of the medicament system, the accessory device may be configured to detect a condition of the delivery device in response to information received by the information detecting and/or receiving component and/or information received via the wired and/or wireless connection with the delivery device, and/or information sensed by the one or more sensors. In one non-limiting example, the accessory device may detect movement of the components of the delivery device by way of a camera, for example, such as detecting the movement of the mechanical components of a metered dose inhaler device when the metered dose inhaler device is actuated, for example. In another non-limiting example, the accessory device may detect movement of or a condition of the delivery device by way of a microphone which listens for sounds generated by the movement(s) of the delivery device and/or its components to indicate, for example, the cap has been removed, a safety shield has been depressed in preparation for an injection with an injection device, in non-limiting embodiments.

In a further embodiment, the attachment component may include a hook, a clip, an adhesive, a holder which may be contoured to receive the delivery device in a non-limiting embodiment, a magnet, or a combination thereof, or any other type of attachment component known to one of ordinary skill in the art. In one non-limiting embodiment, an attachment component may be formed by a portion of the housing of the accessory device, the attachment component may include a surface of the housing, wherein the surface is complementary in shape to the portion of the delivery device upon which it is to be attached, therefore, in one non-limiting example, the attachment component may be snapped onto the delivery device by contact with the surface of the housing of the delivery device and the complementary surface of the housing of the accessory device. In a non-limiting embodiment, the attachment component may be attached to one of the delivery device or the accessory device, and configured to attach the delivery device or the accessory device to the other of the delivery device or the accessory device. For example, the attachment component may be affixed to the accessory device and configure to attach the accessory device to the delivery device, in another non-limiting example. the attachment component may be affixed to the delivery device, and be configured to attach the delivery device to the accessory device. In a further non-limiting example, the attachment component may be a component separate from the delivery device and the accessory device, but may be configure to attach to both of the delivery device and the accessory device.

In the embodiments provided herein, the medicament system embodiments may further comprise a control interface, wherein the control interface may include a responsive member (e.g., switch, button, keypad, etc.) reactive to user input. The control interface may be associated with the delivery device and/or the accessory device in non-limiting embodiments.

Furthermore, as aforementioned herein, the delivery device embodiments described herein may include a medicament training device, a medicament delivery device, or a combination thereof.

In another embodiment, an accessory device configured to receive information from and/or detect information about a delivery device is provided. The accessory device includes a accessory device housing, and may further include a detecting and/or a receiving component, wherein the detecting and/or receiving component is configured to detect information about or receive information from the delivery device, a signal output component may be included, and configured to provide an output to a user, a power source, a microprocessor, and a storage module may be provided, wherein the accessory device may provide a feedback to a user based on information detected and/or received by the detecting and/or receiving component, in a non-limiting embodiment.

In a further embodiment, the accessory device may include a sensor. The sensor may be associated with the accessory device housing, in one embodiment. The sensor may include a proximity sensor, a perpendicularity sensor, a contact sensor, a temperature sensor, a motion sensor, a light sensor, a fluid flow rate sensor, a strain gauge sensor, a pressure sensor, a tilt sensor, a force sensor, a level sensor, a photoelectric sensor, a magnetic sensor, an ultrasonic sensor, an electrochemical sensor, an acceleration sensor, a moisture sensor, a humidity sensor, a speed sensor, an inductive sensor, a capacitive sensor, or an orientation sensor, or a combination thereof, in a non-limiting embodiment.

In one, non-limiting embodiment, the accessory device may include a housing having a top portion, a bottom portion, and an opening for receiving the delivery device. In this particular embodiment, the accessory device may include a first sensor configured to protrude into the opening, wherein a contact between the delivery device and the first sensor indicates that a cap of the delivery device has been removed before placement into the accessory device, as a result of the shape of the distal end of the housing of the delivery device with the cap removed, and the resulting contact between this portion of the delivery device and the first sensor. Therefore, insertion of the delivery device into the accessory device causes information to be detected via the first sensor that the cap has been removed from the delivery device prior to insertion, resulting in activation of the first sensor as the first sensor contacts the delivery device. Further insertion of the delivery device into the accessory device causes the first sensor to move from a first position to a second position, further activating the first sensor as it is moved from the first to the second position as the delivery device is further inserted into the accessory device. In a non-limiting embodiment, the first sensor may include a mechanical switch, or contact switch.

A second sensor may be provided in near, or adjacent to the opening of the accessory device, wherein the second sensor is configured to detect a position of a component of the delivery device, such as a needle shield or other actuator of the delivery device. Further insertion of the delivery device into the accessory device causes the delivery component such as needle shield to contact the bottom portion of the accessory device, movement of the delivery device against the bottom portion of the accessory device causes retraction of the needle shield enabling actuation of the delivery device. The second sensor, in a non-limiting embodiment, may contact the delivery device when the needle shield is in a retracted position. This may be due to the placement of the second sensor as well as the tapered shape of the distal end of the delivery device housing, such that the delivery device housing only contacts the second sensor when the needle shield has been fully retracted. The second sensor may include a contact sensor or a contact switch in non-limiting embodiments. Therefore, activation of the first and second sensors may indicate that the delivery device is activated and ready for use. Feedback may be provided to a user via the delivery device or the accessory device during the interaction between the delivery device and the accessory device before and after the delivery device is activated for use. In one non-limiting embodiment, the accessory device may include an aperture in the bottom portion to allow an injection member to traverse the bottom portion of the accessory device during use of the device. In another embodiment, no aperture is provided.

The accessory device may further include a third sensor on a bottom surface of the accessory device. In another, non limiting embodiment, the third sensor may form a portion of the bottom of the accessory device. In one embodiment, this third sensor may include a capacitive sensor, for example. The third sensor may sense when the accessory device is against the skin of a user. The third sensor may additionally sense when a contact between the safety shield and the third sensor has been made. Consequently, either alone, or in combination with the second sensor, the third sensor may indicate to a user that the delivery device has been fully inserted into the accessory device, the accessory device is against the skin of the user and ready for use, and the safety shield has been retracted into the housing of the delivery device. A combination between contact of the second sensor with the delivery device distal end and contact of the third sensor with the delivery device would indicate the shield has been fully depressed (i.e., retracted), and the injection device is ready for activation.

In a further embodiment, the accessory device may include an internal element that is movable relative to the accessory device. The internal element may include one or more sensors, and may include an opening configured to receive a portion of the delivery device. The internal element may be disposed within the opening of the accessory device, and may receive the delivery device, and be displaced upon movement of the delivery device relative to the accessory device. The accessory device may be placed against the skin of the user, the delivery device distal end may be placed within the opening of the internal element, a spring may be provided to allow displacement and replacement of the internal element relative to the accessory device. Movement of the delivery device into the internal element opening causes activation of contact sensors to indicate removal of the cap of the delivery device and correct placement within the opening, additional sensors may sense contact between the accessory device or the internal element with the skin and with the distal end of the delivery device or the safety shield of the delivery device. These sensors may be provided, as described herein, to detect contact with the delivery device, and depression of the safety shield in preparation for use of the delivery device. Upon movement of the delivery device relative to the accessory device, the internal element is displaced from the accessory device into the skin of the user, the safety shield of the delivery device is depressed, and the delivery device may be actuated. The internal element may include an opening to allow for passage of the injection member of the delivery device to enter a target area on the user skin to deliver an injection. Following the injection, and release of any pressure on the delivery device, the biasing member may extend to retract the internal element into the accessory housing.

In other embodiments described herein, the accessory device may include an attachment component, which may be inherent in the shape and size of a portion of the accessory device, such that it mates with a complementary shape and size of the delivery device, so as to attach onto the delivery device in one, non-limiting embodiment. In other non-limiting embodiments, the attachment component may include an adhesive, a clip, or any other type of attachment component described herein or known to those skilled in the art to attach the accessory device onto the delivery device. In one embodiment, the accessory device may include a contact clement and contact switch, wherein upon movement of the contact element relative to the accessory device, the contact element makes contact with the contact switch to register a status of the delivery device, such as, for example, depression of the safety shield of the device in preparation for an injection. Due to the position of the contact element when the accessory device is attached onto the delivery device, in this non-limiting embodiment, the interaction between the contact element and the contact switch registers that the shield has been deflected when the contact element presses against the contact switch, for example.

In a further embodiment, the accessory device is provided wherein the storage module includes pre-programmed information. The pre-programmed information may include information about a delivery device and/or instructions for use for a delivery device and/or a medicament associated therewith, in a non-limiting example. The accessory device may be further configured to receive or download information about a medicament, a delivery device, and/or a user from the Internet, from another device, and/or from a database, wherein the information can be received or downloaded via a wired and/or a wireless connection.

In still a further embodiment, the accessory device may be configured to provide instructions and/or feedback to the user via the signal output component. In another embodiment, the accessory device may be configured to communicationally connect to the delivery device via a wired and/or a wireless connection to provide communication or transmission of power and/or communication of information between the accessory device and the delivery device.

A communication of power or information can be either one way or two-way. One way communication may occur from the accessory device to the delivery device or vice versa, and two-way communication between the accessory device and the delivery device may occur, in non-limiting embodiments. Communication (or transmission) of power may be from the accessory device to the delivery device in one non-limiting embodiment, from the delivery device to accessory device in another non-limiting embodiment. As aforementioned, the communication may be one way communication or two-way communication between the components of the system. In one particular non-limiting embodiment, information communication is communicated from the delivery device to the accessory device, and power may be communicated from the accessory device to the delivery device.

In an embodiment, the accessory device may associate with a delivery device that includes a mechanism including one or more mechanical components associated with the housing of the delivery device, that move relative to one another to produce a visual, a tactile and/or an audible output detectable by the detecting component of the accessory device. The accessory device may be configured to detect a condition of the delivery device. This detection may be in response to information received by the information detecting and/or receiving component and/or information received via a wired and/or a wireless connection with the delivery device, in a non-limiting embodiment.

In a further non-limiting embodiment, the condition detected may include an error condition, a correct usage of the system, and/or an input sensed by the system. The accessory device may provide feedback to a user of the system via the signal output component based on the condition of the delivery device detected, in a non-limiting embodiment. In a further embodiment, the accessory device may provide instructions for using the delivery device in a sequence of steps, and wherein the microprocessor is configured to control a provision of the instructions for using the delivery device to the user in the sequence of steps and/or to provide an instruction to the user based on information produced by or detected by the sensor of the delivery device. Error conditions may include, in non-limiting examples, putting cap back on a device, an out of sequence operation of a device, a wet injection, and not holding at 90 degrees when required.

In another embodiment, an accessory device configured to receive information from and/or detect information about a delivery device is provided. The accessory device includes an accessory device housing, a sensor associated with the housing; and an attachment component associated with the housing, wherein the attachment component is configured to attach the accessory device to the delivery device. In a further embodiment, the accessory device may include a signal output component configured to provide an output to a user, the output may include information about the delivery device, information about a use of the delivery device, information about a user of the accessory device, and/or information about a medicament, in non-limiting embodiments.

Information communicated between devices may include, for example, compliance information including information about the usage of the system or device. Additional information may include previous uses of the delivery device and/or system, correct and incorrect usage of the device and/or system as well as instructions for use of various delivery devices, contraindications related to various delivery devices and various medicaments, possible medicament interactions, safety information and storage information, reminders or timers identifying next scheduled administration of medicament, dosage information, volume and/or strength of medicament, other instructions regarding medicament including, but not limited to compliance to therapy or treatment, warnings, and any other instructions, among other information typically associated with medicaments and delivery devices. Information may further be communicated between the accessory device and a remote device, such as, for example, a Smartphone, a computer, a database, a PDA, a digital or analog watch, other remote device, or other information receiving device. Information communicated from the accessory device may include information about a use of the delivery device, in a non-limiting embodiment. The remote device may be adapted to receive the information and/or process the information and provide feedback to a user of the system or an additional user such as a medical professional, a family member, or other person or entity. Information may be communicated between the accessory device and one or more remote devices, between the accessory device and the delivery device, or between the medicament system or medicament training device and one or more remote devices. Information can be communicated, as described herein, by way of wired and/or wireless communication. Feedback provided by the remote device may include training information, information about a correct or incorrect usage of the device, system, or medicament, error correction information, positive feedback, reminders regarding using the device, system, or taking a medicament. Other reminders may be provided by the remote device including, but not limited to reminders to make an appointment with a physician, reminders to refill a prescription, reminders to take a medicament, reminders to train using the delivery device, accessory device and/or system, among other reminders.

Information about accessory devices that may be used in conjunction with the embodiments described herein is provided in U.S. Pat. Nos. 10,795,973; 11,081,023; 11,069,260; 11,049,416; 10,784,449; PCT Pub WO2017164927, all of which are incorporated herein in their entirety.

TABLE 1

| Emotional Need | Features |
|---|---|
| Lack of self- | Digital Training Materials |
| confidence | 2D IFU |
| | Videos |
| | Enhanced content, AR/VR etc. |
| | Provide Physical Assets |
| | Mechanical trainers |
| | Error correcting trainers |
| | AdhereIT (training/guidance) |
| | Angle-aid |
| | HCP support sessions |
| | Virtual visits |
| | Appointment scheduling |
| | Personalized treatment plans and roadmaps |
| | Facilitate the formation of rituals in the time |
| | leading up to an injection |
| Fear of side effects | Injection site tracker |
| | Guide user to avoid injecting in the same |
| | site consecutively |
| | Play music as a distraction |
| | Stress reduction techniques |
| | Similar to meditation apps like Headspace |
| | and Calm |
| Stigma of illness/social | Integration with peer support groups and |
| embarrassment | patient-focused social media platforms |
| | Contact caregivers to let them know reaching |
| | out with support would be beneficial |
| | Access to travel kits to enable the activities |
| | users participated in before beginning treatment |
| | Access to AdhereIT to provide a more complete |
| | picture of the injection |
| Frustration with illness | Integration with peer support groups and |
| and treatment | patient-focused social media platforms |
| | HCP support sessions |
| | Virtual visits |
| | Appointment scheduling |
| | Easy/integrated prescription refills |
| | Tracking of symptoms/side effects |
| | Journaling can help remind patients of |
| | improvements |
| | Personalized treatment plants/roadmaps |
| | Track previous treatment experiences |
| Anxiety associated | Integration with peer support groups and patient- |
| with needles | focused social media platforms |
| | Play music as a distraction |
| | Stress reduction techniques (meditation apps) |
| | Provide Physical Assets for practice |
| | Mechanical trainers |
| | Needle simulators |
| | AdhereIT training modes |

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article. As used herein, unless otherwise clear from the context, a value is “about” another value if it is within a factor of two (twice or half) of the other value. While example ranges are given, unless otherwise clear from the context, any contained ranges are also intended in various embodiments. Thus, a range from 0 to 10 includes the range 1 to 4 in some embodiments.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A system for providing personalized self-medication, onboarding, training and improving medication adherence, the system comprising
a patient computer system comprising at least one processor and at least one memory component; a display, an optional input device, and an optional pointing device,
a server connected to the patient computer system by a network link, the server comprising at least one processor and at least one computer readable medium wherein at least one computer readable medium comprises instructions that when executed by at least one processor, cause at least one processor to perform acts comprising:
(i) responsive to patient input data, producing a patient personalized assessment;
(ii) formulating a customized intervention for the patient based on the patient input data and/or personalized assessment; and
(iii) presenting the patient personalized assessment and/or customized intervention on the display.

2. The system of claim 1, wherein the patient input data comprises posology data, environmental factors, treatment history, training usage data, training effectiveness data, emotional state, disease state, physiological data, treatment adherence, diagnostic data, location data, temporal data, and/or treatment effectiveness data.

3. The system of claim 1, wherein the patient input data comprises patient disease state information;

4. The system of claim 1, wherein patient input data comprising emotional state information.

5. The system of claim 1, wherein the patient input data comprises posology information.

6. The system of any of claims 1-5, wherein the patient computer system comprises a network link to a local area network that communicates to the server via an internet connection.

7. The system of any of claims 1-5, wherein the patient computer system is a mobile device that wirelessly communicates with the server via an internet connection.

8. The system of any of claims 1-7, wherein producing a patient personalized assessment comprises producing a personalized assessment of patient training needs and/or patient treatment needs.

9. The system of claim 8, wherein the customized intervention comprises a customized training intervention and/or customized treatment intervention.

10. The system of any of claim 1-5, 6 or 7, wherein producing a patient personalized assessment comprises producing a personalized assessment of patient emotional needs.

11. The system of claim 10, wherein the customized intervention comprises a customized training intervention.

12. The system of any of claim 1-5, 6, or 7 wherein the patient input data comprises training usage data or comprises treatment progress information.

13. The system of claim 12, wherein the at least one memory component of the server further comprises instructions to cause the at least one processor of the server to present on the display a motivational element to the patient in response to training usage data.

14. The system of claim 12, wherein the patient input data is treatment effectiveness data, and further comprising instructions to cause the at least one processor to present on the display a motivational element to the patient in response to treatment effectiveness data.

15. The system of any of claims 1-14, wherein the patient computer system communicates with an accessory device and/or a medicament delivery device via a wired or wireless connection.

16. The system of claim 15, wherein the accessory device is a training device.

17. The system of claim 15, wherein the accessory device is a delivery assistive device.

18. The system of claim 15, wherein the accessory device and/or medicament delivery device comprise at least one sensor.

19. The system of claim 18, wherein the at least one sensor detects operation of the accessory device and/or medicament delivery device.

20. The system of claim 19, wherein the patient input data comprises operation of the accessory device and/or medicament delivery device, or comprises treatment progress information.

21. The system of any of claims 1-20, wherein the server comprises a storage device onto which training videos or training text is stored and wherein the at least one memory component comprises instructions causing the at least one processor of the server to present the training video(s) or training text on the display.

22. The system of claim 21, wherein the server records access and/or presentation of the training videos or training text to the display.

23. The system of claim 22, wherein the at least on memory component causes the at least one processor to present a motivational element on the display in response to access of training video(s) or training text.

24. The system of any of claims 1-23, wherein the server comprises a storage device comprising a plurality assessment variables stored thereon and wherein the at least one memory component of the server comprises instructions that causes the at least one processor to select, in response to patient input data, a combination of assessment variables from the plurality to form the personalized assessment and to present the personalized assessment on the display.

25. The system of any of claims 1-23, wherein the server comprises a storage device comprising a plurality of training variables stored thereon and wherein the at least one memory component of the server comprises instructions that causes the at least one processor to select, in response to patient input data, a combination of intervention variables from the plurality of intervention variables to form the customized intervention and to present the personalized assessment on the display.

26. The system of any of claims 13-25, wherein the server comprises a storage device comprising a plurality of motivational element variables stored thereon and wherein the at least one memory component of the server comprises instructions that causes the at least one processor to select, in response to patient input data, one or more motivational element variables from the plurality of motivational element variables to form the motivational element and to present the motivational element on the display.

27. The system of any of claim 24, 25 or 26, wherein the server comprises a storage device comprising a plurality of surveys stored thereon and wherein the at least one memory component of the server comprises instructions that causes the at least one processor to select, in response to patient input, a survey and to present the survey on the display.

28. The system of claim 18, wherein the at least one sensor is a physiological sensor integral to or connected to an accessory device that comprises circuitry to convey information to the server directly through a wireless connection, or indirectly through the patient computer system or delivery device.

29. The system of claim 28, wherein the physiological sensor is connected wirelessly to the accessory device.

30. The system of claim 15, further comprising at least one sensor that communicates with the patient computer system, accessory device and/or delivery device.

31. The system of claim 30, wherein the at least one sensor comprises a light sensor, fluid flow rate sensor, strain gauge sensor, temperature sensor, pressure sensor, tilt sensor, force sensor, level sensor, contact sensor, photoelectric sensor, magnetic sensor, ultrasonic sensor, electrochemical sensor, acceleration sensor, moisture sensor, humidity sensor, speed sensor, inductive sensor, capacitive sensor, physiological sensor, temporal sensor, geolocation sensor, or orientation sensor.

32. The system of claim 31, wherein the at least one sensor comprises two or more sensors.

33. The system of claim 31, wherein the at least one sensor is integral to the patient computer system.

34. The system of claim 31, wherein the at least one sensor is wirelessly connected to the patient computer system.

35. The system of claim 31, wherein the at least one sensor is integral to the accessory device.

36. The system of claim 31, wherein the at least one sensor is wirelessly connected to the accessory device.

37. The system of claim 31, wherein the at least one sensor is integral to the delivery device.

38. The system of claim 31, wherein the at least one sensor wireless connects to the delivery device.

39. The system of claim 30, wherein the at least one sensor detects errors of mechanical operation of the accessory device and/or delivery device or errors in patient use of the accessory device and/or delivery device.

40. The system of any of claims 1-39, wherein the accessory device comprises a display to convey personalized assessment and/or customized intervention to the patient.

41. The system of any of claims 28-39, wherein information of the at least one sensor is sent to the server via the patient computer system, the accessory device, and/or the delivery device.

42. The system of any of claim 30-39 or 41, wherein the personalized assessment and/or customized intervention is based, at least in part, on information from the sensor.

43. The system of claim 18, wherein the at least one sensor is a geolocation sensor that detects a patient's location during use of an accessory device and/or delivery device.

44. The system of claim 18, wherein the at least one sensor comprises a temporal sensor that detects a time at which a patient uses the accessory device and/or delivery device.

45. The system of claim 43, further comprising a temporal sensor that detects a time at which a patient used the accessory device and/or delivery device.

46. The system of any of claims 43-45, wherein information from the geolocation sensor or temporal sensor, or both, is sent to the server.

47. The system of any of claims 43-45, further comprising at least one physiological sensor, and optionally wherein the at least one physiological sensor provide data to track disease state or disease symptoms.

48. The system of any of claims 43-45 and 47, wherein the at least one sensor further comprises at least one sensor separate to the geolocation sensor and temporal sensor that detects operation of the accessory device and/or delivery device.

49. The system of claim 48, wherein accessory device or patient computer system comprises a camera.

50. A method for providing personalized self-medication training and improving medication adherence executed on a server comprising at least one processor and at least one memory component, the method comprising
receiving patient input data from a patient computer system, the patient computer system comprising at least one processor, at least one memory component and a display;
producing a patient personalized assessment by selecting from a plurality of assessment variables stored on at least one storage device;
formulating a customized intervention for the patient based on the patient input data or personalized assessment; and
presenting the patient personalized assessment and/or customized intervention on the display.

51. The method of claim 50, wherein the patient input data comprises posology data, environmental factors, treatment history, physiological parameters, training usage data, training effectiveness data, emotional state, disease state, treatment adherence, diagnostic data, and/or treatment effectiveness data.

52. The method of claim 50, wherein the patient input data comprises patient disease state information;

53. The method of claim 50, wherein patient input data comprising emotional state information.

54. The method of claim 50, wherein the patient input data comprises posology information.

55. The method of any of claims 50-54 wherein the patient computer system comprises a network link to a local area network that communicates to the server via an internet connection.

56. The method of any of claims 50-54, wherein the patient computer system is a mobile device that wirelessly communicates with the server via an internet connection.

57. The method of any of claims 50-56, wherein producing a patient personalized assessment comprises receiving training information and producing a personalized assessment of patient training needs based on selecting assessment variables in response to training information.

58. The method of claim 57, wherein the customized intervention comprises a customized training intervention or customized treatment intervention.

59. The method of claim 58, wherein the customized treatment intervention comprises notification to change medicament delivery mechanism, medicament delivery frequency, or type of medicament.

60. The method of any of claim 50-54, 55 or 56, wherein producing a patient personalized assessment comprises producing a personalized assessment of patient emotional needs based on selecting assessment variables in response to emotional state/needs information.

61. The method of claim 60, wherein the customized intervention comprises a customized training intervention.

62. The method of any of claim 50-54, 55 or 56, wherein the patient input data comprises training usage information data.

63. The method of claim 62, further comprising presenting on the display a motivational element to the patient in response to training usage data.

64. The method of claim 50-54, 55 or 56, wherein the patient input data comprises treatment progress information.

65. The method of claim 64, further comprising presenting on the display a motivational element to the patient in response to treatment progress information.

66. The method of any of claims 50-65, wherein the patient computer system receives operational information from an accessory device and/or a medicament delivery device optionally via a wired or wireless connection.

67. The method of claim 66, wherein the accessory device is a training device.

68. The method of claim 66, wherein the accessory device is a delivery assistive device.

69. The method of claim 66, 67, or 68, wherein the patient computer system comprises a sensor to detect operation of the accessory device and/or medicament delivery device.

70. The method of claim 69, wherein the sensor comprises a audio detection component or camera whereby that detects sounds and/or movement associated with operation of the accessory device and/or medicament delivery device.

71. The method of claim 66, wherein the accessory device and/or medicament delivery device comprise at least one sensor to detect operation of the accessory device and/or medicament delivery device that is communicated to the patient computer system.

72. The method of claim 69 or 71, wherein the received patient input data comprises the operational information of the accessory device and/or medicament delivery device

73. The method of any of claims 50-72, responsive to patient input, further comprising presenting training video(s) or training text on the display.

74. The method of claim 50, further comprising recording access and/or presentation of the training videos or training text to the display.

75. The method of claim 74, further comprising presenting a motivational element on the display in response to access of training video(s) or training text.
